(19) United States
(12) Patent Application Publication
(10) Pub. No.: US 2024/0181120 A1
(43) Pub. Date: Jun. 6, 2024

(54) FABRICS INCLUDING A BARRIER COATING AND HYGIENE ARTICLES INCLUDING THE SAME

(71) Applicant: Berry Global, Inc., Evansville, IN (US)

(72) Inventors: Ralph A. Moody, III, Mooresville, NC (US); Abhay Joijode, Huntersville, NC (US); Mehmet Selcuk Sinangil, Mooresville, NC (US); Nyle Bishop, Mooresville, NC (US); Lei Wang, Mooresville, NC (US)

(21) Appl. No.: 18/517,058

(22) Filed: Nov. 22, 2023

Related U.S. Application Data
(60) Provisional application No. 63/427,609, filed on Nov. 23, 2022.

(51) Int. Cl. A61L 15/22; A61F 13/49; D06N 3/00; D06N 3/04; D06N 3/12; D06N 3/18

(52) U.S. Cl. CPC A61L 15/225; A61F 13/49017; D06N 3/0011; D06N 3/0084; D06N 3/042; D06N 3/12; D06N 3/125; D06N 3/128; D06N 3/186; A61F 2013/49093; D06N 2201/02; D06N 2201/0254; D06N 2201/047; D06N 2207/14; D06N 2209/14; D06N 2211/00

(57) ABSTRACT

A fabric suitable as an alcohol repellent fabric is provided. The fabric includes a fibrous substrate including a first outermost surface and a second outermost surface, in which a non-fluorinated barrier coating (NFBC) or a fluorinated barrier coating (FBC) is located on at least a portion of a first outermost surface of the fabric, at least a portion of a second outermost surface of the fabric, or both.

### CROSS-REFERENCE TO RELATED APPLICATIONS

This application claims priority under 35 U.S.C. § 119 to U.S. patent application Ser. No. 63/427,609 filed Nov. 23, 2022, which is expressly incorporated by reference herein in its entirety.

### TECHNICAL FIELD

Embodiments of the presently-disclosed invention relate generally to fabrics including a non-fluorinated barrier coating (NFBC) or a fluorinated barrier coating (FBC) located on at least a portion of a first outermost surface of the fabric, in which the NFBC or FBC imparts improved barrier properties in related to low surface tension fluids (e.g., alcohols and blood). The fabrics including the NFBC may provide the same or improved barrier properties as fabrics treated with FBC despite being devoid of fluorine atoms. Hygiene articles including the fabrics are also provided.

### BACKGROUND

Conventional absorbent articles, such as disposable diapers, employ absorbent materials located generally between a liquid pervious topsheet and a liquid impermeable backsheet to absorb body exudates. Conventional absorbent articles, such as diapers, have typically included elasticized waistbands and leg cuffs to help reduce the leakage of body exudates. Some conventional absorbent articles have also included elasticized containment or barrier flaps at the leg or waist sections of the article to further reduce leaks.

The liquid impermeable backsheet on conventional absorbent articles is configured to provide a barrier between the absorbent body within the absorbent article and the wearer's clothes. Typical backsheets have been both liquid and vapor impermeable. For example, many conventional absorbent articles have included a backsheet made from a polymeric film which is both liquid and vapor impermeable.

There, however, remains a need in the art for a nonwoven-based barrier fabric that achieving similar or improved barrier repellency performance with respect to the penetration of low surface tension fluids (e.g., alcohols and blood) as compared to traditional polymeric films.

### SUMMARY OF INVENTION

One or more embodiments of the invention may address one or more of the aforementioned problems. Certain embodiments according to the invention provide a fabric comprising a fibrous substrate including a first outermost surface and a second outermost surface. The fabric may also comprise a non-fluorinated barrier coating (NFBC) located on at least a portion of a first outermost surface of the fabric, at least a portion of a second outermost surface of the fabric, or both. In this regard, the NFBC is devoid of fluorine atoms. In accordance with certain embodiments of the invention, the entirety of the fabric may be devoid of fluorine atoms. In accordance with certain embodiments of the invention, a fluorinated barrier coating (FBC) may be additionally or alternatively located on at least a portion of a first outermost surface of the fabric, at least a portion of a second outermost surface of the fabric, or both.

In another aspect, the present invention provides an absorbent article comprising a fabric having a NFBC and/or FBC, such as those described herein, a liquid permeable topsheet, and an absorbent body located between the fabric and the liquid permeable topsheet.

In another aspect, the present invention provides an absorbent diaper comprising a front region, a back region, an intermediate crotch region located between the front region and the back region. The absorbent diaper may also include at least one barrier leg cuff, in which the at least one barrier leg cuff comprises a fabric having a NFBC and/or FBC, such as those described herein.

In yet another aspect, the present invention provides a method of forming a fabric. The method may comprise the following: (i) providing or forming a fibrous substrate including a first outermost surface and a second outermost surface; (ii) topically applying a fluorinated barrier coating (FBC) composition or a non-fluorinated barrier coating (NFBC) composition on at least a portion of at least the first outermost surface, wherein the NFBC comprises (a) a plurality of radiation curing silicones or (b) a dispersion comprising a solvent, a wax or component thereof, and a retention aid; and (iii) (a) curing the plurality of radiation curing silicones via exposure to radiation, such as ultraviolet (UV) radiation if the NFBC composition comprises the plurality of radiation curing silicones, or (b) actively or passively evaporating the solvent from the dispersion or the FBC composition to provide a fabric having a NFBC and/or FBC, such as those described herein.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1A illustrates a perspective view of an absorbent article according to one embodiment of the invention;

FIG. 1B illustrates a cross-sectional view taken along line X-X in of FIG. 1A; and

FIG. 1C illustrates a perspective view of a portion of the absorbent article of FIG. 1A.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to the use of lower surface tension chemicals such as fluoro-chemicals and/or chemicals that are devoid of fluorine atoms to coat a nonwoven surface (e.g., silicone rubber has a reported surface energy value from 19-22 mJ/m²). In accordance with certain embodiments of the invention, the nonwoven surface may be coated with chemicals that are devoid of fluorine atoms, which may replace the reliance on fluoro-chemicals while achieving similar or improved barrier repellency performance (e.g., prevent the penetration of low surface tension fluids through the fabric). In accordance with certain embodiments of the invention, for example, the fabrics including the NFBC exhibit performance comparable to or better than C6 fluorine chemical treated materials with respect to preventing lower surface tension fluid penetration and also passing the blood drop test with synthetic blood in accordance with ASTM F1670M (e.g., 15 minutes of droplet exposure without blood penetration). By way of example only, polypropylene may have a surface tension of 30.5 mJ/m² and polyethylene may have a surface tension of 31.6 mJ/m². Coating or covering a fabric formed from polypropylene or polyethylene, for example, with the NFBC may reduce the surface tension of the treated surface due to the NFBC, which may render the treated surface resistant to low surface tension fluid penetration.

In accordance with certain embodiments of the invention, the fabric (e.g., a nonwoven fabric) may be made by any method known, such as those described and disclosed herein. Moreover, the fabric may be made from a broad choice of polymeric materials, such as polyolefins (e.g., polypropylene, polyethylene, copolymers thereof, etc.), polyesters, polyamides, natural fibers (e.g., cotton, etc.), and cellulosic fibers (e.g., rayon, wood fibers, etc.). In accordance with certain embodiments of the invention, for example, the fabric comprises a nonwoven comprising polyolefin thermoplastic polymer. For example, the nonwoven fabric may comprise a polypropylene (e.g., polypropylene being defined broadly and includes copolymers and blends containing a polypropylene). In accordance with certain embodiments of the invention, the nonwoven fabric may comprise a polyethylene (e.g., polyethylene monocomponent fibers, bi-component fibers including a polyethylene component, flash spun polyethylene fibers, etc.).

In accordance with certain embodiments of the invention, the nonwoven fabric may comprise continuous fibers (e.g., spunbond fibers), staple fibers, fine fibers (e.g., defined broadly to include melt-blown, melt-film fibrillated, electrospun, etc.). As noted above, certain embodiments of the invention may comprise a nonwoven fabric comprising a layer of cellulosic fibers (e.g., wood pulp) and a layer of synthetic fibers (e.g., thermoplastic polymer) mechanically entangled together (e.g., hydroentangled together). In accordance with certain embodiments of the invention, the nonwoven fabric may comprise continuous fibers (e.g., spunbond fibers) and fine fibers (e.g., meltblown fibers), such as fabrics having a spunbond-meltblown-spunbond (SMS), such as a SMS or SSMMS structure, where one or several layers of meltblown fibers are sandwiched in between layers of continuous fibers.

The terms “substantial” or “substantially” may encompass the whole amount as specified, according to certain embodiments of the invention, or largely but not the whole amount specified (e.g., 95%, 96%, 97%, 98%, or 99% of the whole amount specified) according to other embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantionmers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The terms “nonwoven” and “nonwoven web”, as used herein, may comprise a web having a structure of individual fibers, filaments, and/or threads that are interlaid but not in an identifiable repeating manner as in a knitted or woven fabric. Nonwoven fabrics or webs, according to certain embodiments of the invention, may be formed by any process conventionally known in the art such as, for example, meltblowing processes, spunbonding processes, needle-punching, hydroentangling, air-laid, and bonded carded web processes. A “nonwoven web”, as used herein, may comprise a plurality of individual fibers that have not been subjected to a consolidating process. In certain instances, the “nonwoven web” may comprises a plurality of layers, such as one or more spunbond layers and/or one or more meltblown layers. For instance, a “nonwoven web” may comprises a spunbond-meltblown-spunbond structure.

The terms “fabric” and “nonwoven fabric”, as used herein, may comprise a web of fibers in which a plurality of the fibers are mechanically entangled or interconnected, fused together, and/or chemically bonded together. For example, a nonwoven web of individually laid fibers may be subjected to a bonding or consolidation process to bond at least a portion of the individually fibers together to form a coherent (e.g., united) web of interconnected fibers.

The term “consolidated” and “consolidation”, as used herein, may comprise the bringing together of at least a portion of the fibers of a nonwoven web into closer proximity or attachment there-between (e.g., thermally fused together, chemically bonded together, and/or mechanically entangled together) to form a bonding site, or bonding sites, which function to increase the resistance to external forces (e.g., abrasion and tensile forces), as compared to the unconsolidated web. The bonding site or bonding sites, for example, may comprise a discrete or localized region of the web material that has been softened or melted and optionally subsequently or simultaneously compressed to form a discrete or localized deformation in the web material. Furthermore, the term “consolidated” may comprise an entire nonwoven web that has been processed such that at least a portion of the fibers are brought into closer proximity or attachment there-between (e.g., thermally fused together, chemically bonded together, and/or mechanically entangled together), such as by thermal bonding or mechanical entanglement (e.g., hydroentanglement) as merely a few examples. Furthermore, the term “consolidated” and “consolidation” may comprise the bonding by means of a through-air-bonding operation. The term “through-air bonded” and “though-air-bonding”, as used herein, may comprise a nonwoven web consolidated by a bonding process in which hot air is used to fuse the fibers at the surface of the web and optionally internally within the web. By way of example only, hot air can either be blown through the web in a conveyorized oven or sucked through the web as it passes over a porous drum as a vacuum is developed. The temperature of and the rate of hot air are parameters that may determine the level or the extent of bonding in nonwoven web. In accordance with certain embodiments of the invention, the temperature of the hot air may be high enough to melt, induce flowing, and/or fuse the a plurality of fibers having a lower melting point temperature or onset of lower melting point temperature (e.g., amorphous fibers) to a plurality of fibers having a higher melting point temperature or onset of lower melting point temperature (e.g., semi-crystalline or crystalline fibers). Such a web may be considered a “consolidated nonwoven”, “nonwoven fabric” or simply as a “fabric” according to certain embodiments of the invention.

The term “layer”, as used herein, may comprise a generally recognizable combination of similar material types and/or functions existing in the X-Y plane.

The term “spunbond”, as used herein, may comprise fibers which are formed by extruding molten thermoplastic material as filaments from a plurality of fine, usually circular, capillaries of a spinneret with the diameter of the extruded filaments then being rapidly reduced. According to an embodiment of the invention, spunbond fibers are generally not tacky when they are deposited onto a collecting surface and may be generally continuous as disclosed and described herein. It is noted that the spunbond used in certain composites of the invention may include a nonwoven described in the literature as SPINLACE®.

As used herein, the term “continuous fibers” refers to fibers which are not cut from their original length prior to being formed into a nonwoven web or nonwoven fabric. Continuous fibers may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “meltblown”, as used herein, may comprise fibers formed by extruding a molten thermoplastic material through a plurality of fine die capillaries as molten threads or filaments into converging high velocity, usually hot, gas (e.g. air) streams which attenuate the filaments of molten thermoplastic material to reduce their diameter, which may be to microfiber diameter, according to certain embodiments of the invention. According to an embodiment of the invention, the die capillaries may be circular. Thereafter, the meltblown fibers are carried by the high velocity gas stream and are deposited on a collecting surface to form a web of randomly disbursed meltblown fibers. Meltblown fibers may comprise microfibers which may be continuous or discontinuous and are generally tacky when deposited onto a collecting surface. Meltblown fibers, however, are shorter in length than those of spunbond fibers.

The term “melt fibrillation”, as used herein, may comprise a general class of making fibers defined in that one or more polymers are molten and may be extruded into many possible configurations (e.g. co-extrusion, homogeneous or bicomponent films or filaments) and then fibrillated or fiberized into a plurality of individual filaments for the formation of melt-fibrillated fibers. Non limiting examples of melt-fibrillation methods may include melt blowing, melt fiber bursting, and melt film fibrillation. The term “melt-film fibrillation”, as used herein, may comprise a method in which a melt film is produced from a melt and then a fluid is used to form fibers (e.g., melt-film fibrillated fibers) from the melt film. Examples include U.S. Pat. Nos. 6,315,806, 5,183,670, 4,536,361, 6,382,526, 6,520,425, and 6,695,992, in which the contents of each are incorporated by reference herein to the extent that such disclosures are consistent with the present disclosure. Additional examples include U.S. Pat. Nos. 7,628,941, 7,722,347, 7,666,343, 7,931,457, 8,512,626, and 8,962,501, which describe the Arium™ melt-film fibrillation process for producing melt-film fibrillated fibers (e.g., having sub-micron fibers).

As used herein, the term “aspect ratio”, comprise a ratio of the length of the major axis to the length of the minor axis of the cross-section of the fiber in question.

The term “multi-component fibers”, as used herein, may comprise fibers formed from at least two different polymeric materials or compositions (e.g., two or more) extruded from separate extruders but spun together to form one fiber. The term “bi-component fibers”, as used herein, may comprise fibers formed from two different polymeric materials or compositions extruded from separate extruders but spun together to form one fiber. The polymeric materials or polymers are arranged in a substantially constant position in distinct zones across the cross-section of the multi-component fibers and extend continuously along the length of the multi-component fibers. The configuration of such a multi-component fiber may be, for example, a sheath/core arrangement wherein one polymer is surrounded by another, an eccentric sheath/core arrangement, a side-by-side arrangement, a pie arrangement, or an “islands-in-the-sea” arrangement, each as is known in the art of multicomponent, including bicomponent, fibers.

The term “fluorochemical”, as used herein, may comprise any of various chemical compounds containing fluorine, particularly organic compounds (e g., fluorocarbons such as perfluoroalkanes) in which fluorine has replaced a large proportion of the hydrogen attached to the carbons Fluorochemicals may exhibit low surface tension and low viscosity and are extremely stable due to the strength of the carbon-fluorine bond. Fluorochemicals are not miscible with most organic solvents.

The term “dry basis”, as used herein may comprise the calculation or measurement of a weight percentage in which the presence of water and/or other solvents (e.g., alcohols) are ignored or excluded for purposes of the calculation or measurement. Weight percentages may frequently be measured on a dry basis to remove the effects of evaporation and/or condensation which may happen naturally throughout the useful life of a composition or article.

The term “cellulosic fiber”, as used herein, may comprise fibers derived from hardwood trees, softwood trees, or a combination of hardwood and softwood trees prepared for use in, for example, a papermaking furnish and/or fluff pulp furnish by any known suitable digestion, refining, and bleaching operations. The cellulosic fibers may comprise recycled fibers and/or virgin fibers. Recycled fibers differ from virgin fibers in that the fibers have gone through the drying process at least once. In certain embodiments, at least a portion of the cellulosic fibers may be provided from non-woody herbaceous plants including, but not limited to, kenaf, cotton, hemp, jute, flax, sisal, or abaca. Cellulosic fibers may, in certain embodiments of the invention, comprise either bleached or unbleached pulp fiber such as high yield pulps and/or mechanical pulps such as thermo-mechanical pulping (TMP), chemical-mechanical pulp (CMP), and bleached chemical-thermo-mechanical pulp BCTMP. In this regard, the term “pulp”, as used herein, may comprise cellulose that has been subjected to processing treatments, such as thermal, chemical, and/or mechanical treatments. Cellulosic fibers, according to certain embodiments of the invention, may comprise one or more pulp materials.

All whole number end points disclosed herein that can create a smaller range within a given range disclosed herein are within the scope of certain embodiments of the invention. By way of example, a disclosure of from about 10 to about 15 includes the disclosure of intermediate ranges, for example, of: from about 10 to about 11; from about 10 to about 12; from about 13 to about 15; from about 14 to about 15; etc. Moreover, all single decimal (e.g., numbers reported to the nearest tenth) end points that can create a smaller range within a given range disclosed herein are within the scope of certain embodiments of the invention. By way of example, a disclosure of from about 1.5 to about 2.0 includes the disclosure of intermediate ranges, for example, of: from about 1.5 to about 1.6; from about 1.5 to about 1.7; from about 1.7 to about 1.8; etc.

Certain embodiments according to the invention provide a fabric comprising a fibrous substrate including a first outermost surface and a second outermost surface. The fabric may also comprise a non-fluorinated barrier coating (NFBC) located on at least a portion of a first outermost surface of the fabric, at least a portion of a second outermost surface of the fabric, or both. In this regard, the NFBC is devoid of fluorine atoms. In accordance with certain embodiments of the invention, the entirety of the fabric may be devoid of fluorine atoms. In accordance with certain embodiments of the invention, a fluorinated barrier coating (FBC) may be additionally or alternatively located on at least a portion of a first outermost surface of the fabric, at least a portion of a second outermost surface of the fabric, or both. For example, the NFBC and/or the FBC may be located on at least a portion of the first outermost surface and also on at least the second outermost surface. The first outermost surface may have the NFBC on one or more separate and discrete locations or, alternatively, be completely coated with the NFBC. In accordance with certain embodiments of the invention, the second outermost surface may have the NFBC on one or more separate and discrete locations or, alternatively, be completely coated with the NFBC. In accordance with certain embodiments of the invention, the first outermost surface may include an FBC, while the second outermost surface may include the NFBC. In accordance with certain embodiments of the invention, the fabric (e.g., nonwoven fabric) may be devoid of the FBC.

In accordance with certain embodiments of the invention, the FBC (if present) comprise at least one fluorochemical comprising at least one C4 fluorochemical, a C6 fluorochemical, a C8 fluorochemical, a C10 fluorochemical, or any combination thereof. Although the FBC may be applied via a variety of topical application operations, such as spraying, kiss-rolling, and submersion in a bath, the FBC may also comprise a digitally printed or sprayed composition comprising a plasma cured composition including at least one fluorochemical comprising at least one C4 fluorochemical, a C6 fluorochemical, a C8 fluorochemical, a C10 fluorochemical, or any combination thereof. For example, a digitally printed or sprayed FBC may comprise a plasma cured composition; wherein the plasma cured composition comprises a polymerized composition of a free-radically curable monomeric composition including at least a first group of monomers including at least one free-radically polymerizable functional group and at least one side chain comprising from about 4 to about 10 fluorinated carbon atoms, such as 4, 5, 6, 7, 8, 9, or 10 fluorinated carbon atoms. The digitally printed or sprayed FBC, for example, may comprise a plasma cured composition, wherein the plasma cured composition comprises a polymerized composition including a perfluoroalkylethylmethacrylate containing at least one side chain from about 4 to about 10 fluorinated carbon atoms, such as 4, 5, 6, 7, 8, 9, or 10 fluorinated carbon atoms.

In accordance with certain embodiments of the invention, the FBC may be formed from a FBC composition including a fluorochemical compound or compounds. While in a pre-cured state, the FBC composition may include one or monomers that that include one or more fluorochemical chains and one or more polymerizable functional groups (e.g., free-radically polymerizable functional groups that polymerize by the action of free radicals). For instance, the one or more fluorochemical chains may impart the desired alcohol repellency in the cured composition, while the one or more polymerizable functional groups enable, for example, a radically-mediated curing of the pre-cured alcohol repellent composition, such as upon exposure to one or more atmospheric plasma curing operations. Although the polymerizable functional groups are not particularly limited, acrylate and/or methacrylate groups among other groups having double and/or triple bonds may be used in accordance with certain embodiments of the invention. For instance, the polymerizable functional groups may comprise an acrylate or methacrylate group, an allylic group, an alkyne group, a styrenic group, a vinyl ether group, a vinyl ester group, a vinyl amide group, a maleate group, a fumarate group, a crotonate group, a cinnamate group, or a norbornene group. After being applied to the fibrous structure, the pre-cured FBC composition may be moved into the vicinity of plasma treatment regions such that excited species therefrom impinge thereon. The polymerizable monomers cure upon exposure to the plasma treatment forming a cured alcohol repellent composition that adheres to the fibrous structure. As an example, the hydrocarbon portions of polymerized 2-(Perfluorohexyl) ethyl acrylate (commonly referred to as C6) bond to each other and to the fibrous structure, while the fluorinated chains face away from the fibrous structure and repel alcohol, water, and/or oil. In accordance with certain embodiments of the invention, the pre-cured and/or cured FBC comprises less than about 5% by weight of water, less than about 3% by weight of water, less than about 2% by weight of water, or less than about 1% by weight of water, or less than about 0.5% by weight of water. In accordance with certain embodiments of the invention, the pre-cured and/or cured FBC is devoid of water.

In accordance with certain embodiments of the invention, the first outermost surface, the second outermost surface, or both may comprise at least a portion thereof coated with a combination of a FBC and a NFBC. For example, a ratio between the FBC to the NFBC may comprise from about 0.1:1 to about 10:1, such as at least about any of the following: 0.1:1, 0.3:1, 0.5:1, 0.8:1, 1:1, 1.5:1, 2: 1, 2.5:1, 3:1, 3.5:1, 4:1, 4.5:1 and 5:1, and/or at most about any of the following: 10:1, 9:1, 8:1, 7:1, 6:1, and 5:1.

In accordance with certain embodiments of the invention, the NFBC may be located on at least the first outermost surface and located in one or more treated discrete area. For example, the one or more discrete treated areas may cover from about 1% to about 100% of the first outermost surface, such as at least about any of the following: 1, 3, 5, 10, 15, 20, 25, 30, 35, 40, 45, and 50% and/or at most about any of the following: 100, 99, 95, 90, 85, 80, 75, 70, 65, 60, 55, and 50%. Additionally or alternatively, the second outermost surface may comprise the NFBC located in one or more treated discrete area of the second outermost surface. For example, the one or more discrete treated areas of the second outermost surface may cover from about 1% to about 100% of the second outermost surface, such as at least about any of the following: 1, 3, 5, 10, 15, 20, 25, 30, 35, 40, 45, and 50% and/or at most about any of the following: 100, 99, 95, 90, 85, 80, 75, 70, 65, 60, 55, and 50%. In accordance with certain embodiments of the invention, for example, the NFBC may comprise a first NFBC comprising a first NFBC composition located on the first outermost surface of the fabric and a second NFBC comprising a second NFBC composition located on the second outermost surface. The first NFBC composition and the second NFBC composition may be identical to each other or alternatively be different from each other.

In accordance with certain embodiments of the invention, the fabric may comprise an antistatic composition located on the first outermost surface and the NFBC may be located on the second outermost surface. In accordance with certain embodiments of the invention, the antistatic composition may be located on at least a portion of the first outermost surface and also on at least the second outermost surface. The first outermost surface may have the antistatic composition on one or more separate and discrete locations or, alternatively, be completely coated with the antistatic composition. In accordance with certain embodiments of the invention, the second outermost surface may have the antistatic composition on one or more separate and discrete locations or, alternatively, be completely coated with the antistatic composition. In accordance with certain embodiments of the invention, the NFBC and the antistatic composition may be applied separately to the fibrous substrate and/or applied to different regions of the fibrous substrate, whether on the same side of the fibrous substrate or different sides of the fibrous substrate. Additionally or alternatively, the NFBC may incorporate the antistatic composition (e.g., formulated as a single composition including the constituents of both the NFBC and the antistatic composition). In this regard, a single composition may provide a method of simultaneously applying a NFBC and an antistatic agent.

In accordance with certain embodiments of the invention, the antistatic composition comprises at least one antistatic agent, in which the antistatic composition comprises at least one of a non-ionic antistatic agent, an anionic antistatic agent, a cationic antistatic agent, an amphoteric antistatic agent, or any combination thereof. In accordance with certain embodiments of the invention, the at least one antistatic agent comprises an alkylphosphate or a phosphate ester.

In accordance with certain embodiments of the invention, the antistatic composition may comprise from about 0.01 to about 10% by weight (e.g., on a dry basis) of the fabric, such as at least about 0.01, 0.05, 0.1, 0.25, 0.5, 0.75, 1, 1.5, 2, 2.5, 3, 3.5, 4, 4.5, and 5% by weight (e.g., on a dry basis) of the fabric and/or at most about any of the following: 10, 9, 8, 7, 6, and 5% by weight (e.g., on a dry basis) of the fabric.

In accordance with certain embodiments of the invention, the NFBC comprises a cured siliconized coating. For example, the cured siliconized coating may comprise a network of radiation cured silicones. The network of radiation cured silicones may be cured and/or bonded to the fibrous structure via one of more radically curing functionalities. For example, the network of radiation cured silicones may comprise the reaction product of one or more silicone acrylate oligomers or polymers or one or more epoxy silicone oligomers or polymers.

In accordance with certain embodiments of the invention, the one or more silicone acrylate oligomers or polymers are selected from Formula (I):

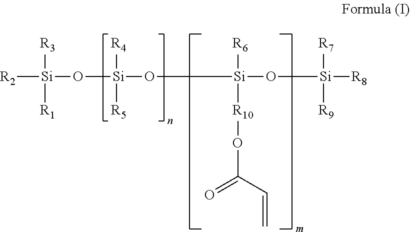

wherein,

R1-R9 are independently selected from —H, a C1-C10 radical, —OH, alkoxy radical, and an acrylate functional group,

R10 is selected from a C1-C10 hydrocarbon;

n is selected from 1 to about 100, such as at least about any of the following: 1, 3, 5, 10, 20, 30, 40, and 50, and/or at most about any of the following: 100, 90, 80, 70, 60, and 50, and

m is selected from 1 to about 100, such as at least about any of the following: 1, 3, 5, 10, 20, 30, 40, and 50, and/or at most about any of the following: 100, 90, 80, 70, 60, and 50.

In accordance with certain embodiments of the invention the acrylate group, for example, from Formula (I) may be a methacrylate group. Additionally or alternatively, one or more of R1-R10 may include an acrylate or methacrylate group.

In accordance with certain embodiments of the invention, the one or more epoxy silicone oligomers or polymers are selected from Formula (II):

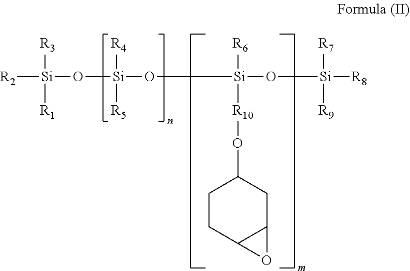

wherein

R1-R9 are independently selected from —H, a C1-C10 radical, —OH, alkoxy radical, and an acrylate functional group,

R10 is selected from a C1-C10 hydrocarbon;

n is selected from 1 to about 100, such as at least about any of the following: 1, 3, 5, 10, 20, 30, 40, and 50, and/or at most about any of the following: 100, 90, 80, 70, 60, and 50, and

m is selected from 1 to about 100, such as at least about any of the following: 1, 3, 5, 10, 20, 30, 40, and 50, and/or at most about any of the following: 100, 90, 80, 70, 60, and 50.

In accordance with certain embodiments of the invention, the NFBC may comprise a coating composition including (a) a wax or a component thereof having an acid value of from 10 mg to 220 mg, KOH/g as measured in accordance with USP 401, such as at least about any of the following: 10, 15, 20, 30, 40, 50, 60, 80, and 100 mg, KOH/g as measured in accordance with USP 401 and/or at most about any of the following: 220, 200, 180, 160, 150, 140, 120, and 100 mg, KOH/g as measured in accordance with USP 401 and (b) a retention aid comprising a nitrogen-containing polymer independently selected from the group consisting of:

(i) a nitrogen-containing polymer of Formula (III),

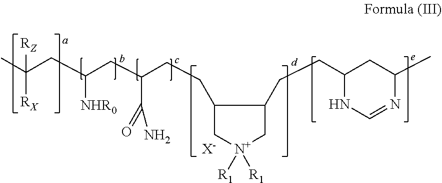

wherein ‘a’, ‘b, ‘c’, ‘d’, and ‘e’ individually represent the molar percent of each repeating unit included in the nitrogen-containing polymer of Formula (III),

wherein R₀ is independently selected from the group consisting of: of H,

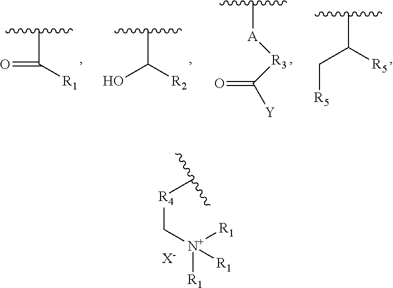

and combinations thereof, and

wherein:
R_{z} is independently selected from H, —CH₃, and combinations thereof,
Rₓ is independently selected from H, —OH, —COOH, —COOR₁, —OCOR₁, —R₁, —R₃OH, —OR₁, —NR₁R₁, —R₃NH₂, —NH₂, —COO(CH₂)₂N(R₁)₂, —COO(CH₂)₂N+(R₁)₃X⁻, —COO(CH₂)₃N+(R₁)₃X⁻, and combinations thereof, with the proviso that when Rₓ is —NH₂, R_{z} is —CH₃,
Y is independently selected from H, —OH, —R₁, —OR₁, —NR₁R₁, —NH₂, and combinations thereof,
Z is independently selected from H, —OH, —C═O, —R₁, —OR₁, —NR₁R₁, —NH₂ and combinations thereof,
R₁ is independently selected from H, a straight chain or branched alkyl or alkenyl containing up to 22 carbons, and combinations thereof,
R2 is independently selected from H, a monosaccharide, an oligosaccharide, polysaccharide moiety, a straight or branched alkyl or alkenyl group up to 22 carbons optionally containing a hydroxyl or aldehyde group, and combinations thereof,
R₃ is independently selected from a straight chain or branched alkyl or alkenyl containing up to 22 carbons or combinations thereof,
R₄ is independently selected from a straight chain or branched alkyl group containing up to 18 carbons, optionally substituted with a hydroxyl group, and combinations thereof,
R₅ is independently selected from H, —OH, —COOH, —COOR₁, —OCOR₁, —R₁, —R₃OH, —OR₁, —CONH₂, —CONHCHOHCHO, —NR₁, —NR₁R₁, —R₁NH₂, —NH₂, and combinations thereof,
A is independently selected from C═O, —CH₂, and combinations thereof, and
X⁻ is independently an anion;
ii. a polyethyleneimine;
iii. a polyaminoamide;
iv. a copolymer formed from the reaction product of epichlorohydrin and dimethylamine;
   and
v. combinations thereof.

In accordance with certain embodiments of the invention, ‘a’, ‘b, ‘c’, ‘d’, and ‘e’ of Formula (III) may each independently from each other have a value from 0 to 100 mol. %, such as at least about any of the following: 0, 1, 3, 5 ,8, 10, 15, 20, 25, 30, 35, 40, 45, and 50 mol. %, and/or at most about any of the following: 100, 95, 90, 85, 80, 75, 70, 65, 60, 55, and 50 mol. %.

Persons having ordinary skill in the art understand that many waxes, particularly naturally occurring waxes, include a combination of individual components. For example, naturally occurring beeswax includes palmitate, palmitoleate, and oleate esters of long-chain (e.g. 30-32 carbons) aliphatic alcohols, with each individual component being a “component thereof” in relation to beeswax. For ease of reference, the term “wax or component thereof” may be collectively referred to as “wax” throughout the remaining description.

Although the wax may not be limited to any particular wax in accordance with certain embodiments of the invention, provided the wax has an acid value from 10 mg to 220 mg, KOH/g, typically the wax may be selected from a group consisting of a stearate, beeswax (both synthetic and natural), candelilla wax, palmitate, behenate, and combinations thereof. For example, the wax of the sizing agent may be beeswax or a stearate, or both. Alternatively, the wax may be behenate or palmitate, or both.

In accordance with certain embodiments of the invention, the coating composition may include any composition disclosed in U.S. Ser. No. 17/498,221 (i.e., U.S. Publication No. 2022/01788078), for example as a component of the NFBC, assigned to AGC Chemicals Americas, Inc. (PA, USA), the contents of which are hereby incorporated by reference to the extent its disclosure is consistent with the aims of embodiments of the present invention.

In accordance with certain embodiments of the invention, the NFBC may comprise from about 0.05 to about 20% by weight on a dry basis of the fabric, such as at least about any of the following: 0.05, 0.08, 0.1, 0.3, 0.5, 0.8, 1, 1.2, 1.5, 1.6, 1.8, 2, 3, 4, and 5% by weight on a dry basis, and/or at most about any of the following: 20, 18, 15, 12, 10, 8, 6, and 5% by weight on a dry basis.

In accordance with certain embodiments of the invention, the fibrous substrate has a basis weight from about 5 to about 200 grams-per-square meter (gsm), such as at least about any of the following: 5, 8, 10, 12, 15, 18, 20, 30, 40, 50, 60, 70, 80, 90, and 100 gsm and/or at most about any of the following: 200, 175, 150, 125, and 100 gsm. Additionally or alternatively, the fabric (e.g., fibrous substrate plus NFBC and any other additives) may have a basis weight from about 5 to about 200 gsm, such as at least about any of the following: 5, 8, 10, 12, 15, 18, 20, 30, 40, 50, 60, 70, 80, 90, and 100 gsm and/or at most about any of the following: 200, 175, 150, 125, and 100 gsm.

In accordance with certain embodiments of the invention, the fabric has a first ratio between the NFBC (% weight of the fabric on a dry basis) to the antistatic composition (% weight of the fabric on a dry basis) or at least one antistatic agent from about 0.2:1 to about 3:1, such as at least about any of the following: 0.2:1 , 0.4:1, 0.6:1, 0.8:1, 1:1 and/or at most about any of the following: 3:1, 2.5:1, 2:1, 1.5:1, and 1:1. In accordance with certain embodiments of the invention, the at least one antistatic agent may comprise from about 0.01 to about 0.5% by weight on a dry basis of the fabric, such as at least about any of the following: 0.01, 0.02, 0.05, 0.08, 0.1, 0.2, and 0.25% by weight on a dry basis of the fabric, and/or at most about any of the following: 0.5, 0.4, 0.3, and 0.25% by weight on a dry basis of the fabric. In accordance with certain embodiments of the invention, the fabric may be devoid of any antistatic agent.

In accordance with certain embodiments of the invention, the fibrous substrate may comprise one or more woven materials, one or more nonwoven materials, one or more film layers, one or more natural and/or synthetic cellulose layers (e.g., pulp, paper, tissue, etc.), or any combination thereof. In accordance with certain embodiments of the invention, the one or more nonwoven materials may comprise one or more spunbond layers, one or more meltblown layers, one or more melt-fibrillated layers, one or more electrospun layers, one or more carded nonwoven layers, one or more hydroentangled layers, or any combinations thereof. For example, the fibrous substrate may comprise a SMS, S, SS, SSS, Meltblown itself or hydro-entangled fiber or pulp alone or in combination with any of the nonwoven layer or layers described and disclosed herein.

In accordance with certain embodiments of the invention, the fibrous substrate may comprise both cellulosic and synthetic fibers. For example, the fibrous substrate may comprise one or more physically entangled (e.g., hydroentangled) nonwoven layers comprising synthetic (e.g., thermoplastic) fibers alone or in combination with cellulosic fibers (e.g., pulp, rayon, viscose, etc.) The fibrous substrate, for instance, may include a physically entangled (e.g., hydroentangled) nonwoven layer including continuous spunbond fibers, which may comprise a polyolefin such as a polypropylene, and cellulosic fibers or pulp. By way of example only, one or more spunbond layers and one or more cellulosic layer (e.g., air-laid pulp layer, tissue layer, etc.) may be stacked and subjected to hydroentanglement to physically consolidate the spunbond and the cellulosic fibers into a single nonwoven layer.

In accordance with certain embodiments of the invention, the fibrous structure may comprise one of the following structures:
(Structure 1) S1ₐ-M_{b}-S2_{c};
(Structure 2) S1ₐ-N_{d}-S2_{c};
(Structure 3) S1ₐ-M_{b}-N_{d}-S2_{c};
(Structure 4) S1ₐ-N_{d}-S3ₑ-N_{d}-S2_{c};
(Structure 5) S1ₐ-N_{d}-M_{b}-N_{d}-S2_{c};
(Structure 6) S1ₐ-M_{b}-S3ₑ-M_{b}-S2_{c};
(Structure 7) S1ₐ-M_{b}-N_{b}-M_{b}-S2_{c}; or any combinations thereof;

wherein
‘M’ comprises a meltblown layer or melt-film fibrillated layer;
‘N’ comprises a sub-micron fiber-containing layer;
‘S1’ comprises a first spunbond layer;
‘S2’ comprises a second spunbond layer;
‘S3’ comprises a third spunbond layer;
‘a’ represents the number of layers and is independently selected from 1, 2, 3, 4, and 5;
‘b’ represents the number of layers is independently selected from 1, 2, 3, 4, 5, 6, 7, and 8;
‘c’ represents the number of layers is independently selected from 1, 2, 3, 4, and 5; and
‘d’ represents the number of layers is independently selected from 1, 2, 3, 4, and 5;
‘e’ represents the number of layers is independently selected from 1, 2, 3, 4, and 5.

The fibrous structure, in accordance with certain embodiments of the invention, may comprise one or more layers containing a plurality of cellulosic fibers, in which the plurality of cellulosic fibers comprises a plurality of natural synthetic fibers, a plurality of synthetic cellulosic fibers, or combinations thereof. In accordance with certain embodiments of the invention, the plurality of cellulosic fibers may be physically entangled with a plurality of spunbond fibers, a plurality of meltblown fibers, a plurality of staple fibers, or any combination thereof. As noted above, the fibrous structure may be physically entangled (e.g., hydroentangled) with a variety of layers, including any fibrous structures according to Structures 1-7.

In accordance with certain embodiments of the invention, the fibrous structure may comprise from about 0 to about 60% by weight of meltblown fibers, such as at least about any of the following: 0, 5, 10, 15, 20, 25, 30, and 35% by weight and/or at most about any of the following: 60, 55, 50, 45, 40, and 35% by weight.

In accordance with certain embodiments of the invention, the fabric may comprise at least one binder. The at least one binder (if present), for instance, may comprise an anionic binder, a cationic binder, non-ionic binder, an amphoteric binder, or any combinations thereof. The binder, in accordance with certain embodiments of the invention, may comprise binder agents that are self-cross linking chemicals (e.g., self-crosslinking non-ionic binder) that improve barrier properties, particularly when formulated with the NFBC. In this regard, for example, the NFBC may contain one or more binding agents to the fabric via the same application time and/or operation. In accordance with certain embodiments of the invention, the binder may comprise an ethylene vinyl acetate copolymer emulsion, an acrylic emulsion, a vinyl acrylic emulsion, or combinations thereof. For example, the at least one binder may comprise at least one of an acrylic binder, a styrene-butadiene rubber binder, a vinyl copolymer binder, a vinyl acetate binder, an ethylene vinyl acetate binder, a polyvinyl chloride binder, a polyurethane binder, or any combination thereof. In accordance with certain embodiments of the invention, the at least one binder comprises an acrylic binder, such as an anionic acrylic binder, a cationic acrylic binder, or a non-ionic acrylic binder. The binder agent(s), in accordance with certain embodiments of the invention, may comprise from about 0.01 to about 50% by weight on a dry basis of the fabric, such as at least about 0.01, 0.05, 0.1, 0.2, 0.25, 0.5, 0.75, 1, 1.5, 2, 2.5, 3, 3.5, 4, 4.5, 5, 6, 8, and 10% by weight on a dry basis of the fabric and/or at most about any of the following: 50, 40, 30 20, 18, 15, 12, and 10% by weight on a dry basis of the fabric. The inclusion of a binder agent(s), in accordance with certain embodiments of the invention, may improve the hydrohead and/or the alcohol repellency of the fabric. Additionally or alternatively, the binder on a dry basis may account for about 10 to about 60% by weight of the NFBC, such as at least about any of the following: 10, 15, 20, 25, and 30% by weight of the NFBC on a dry basis, and/or at most about any of the following: 60, 58, 55, 52, 40, 48, 45, 42, 40, 38, 35, 32, and 30% by weight of the NFBC on a dry basis. Additionally or alternatively, the NFBC may have a ratio on a dry basis between all solids content of the NFBC other than the binder to the solids content of the binder from about 0.5:1 to about 8:1, such as at least about any of the following: 0.5:1, 0.8:1, 1:1, 1.2:1, 1.5:1, 1.8: 1, 2: 1, 2.2: 1, 2.5:1, 2.8:1, and 3:1, and/or at most about any of the following: 8:1, 7.5:1, 7:1, 6.5:1, 6:1, 5.8:1, 5.5:1, 5.2:1, 5:1, 4.8:1, 4.5:1 4.2:1, 4:1, 3.8:1, 3.5:1, 3.2:1, and 3:1. In accordance with certain embodiments of the invention, however, the fabric may be devoid of a binder.

In accordance with certain embodiments of the invention, the fibrous substrate comprises one or more spunbond layers, one or more meltblown layers, one or more melt-fibrillated layers, one or more electrospun layers, one or more carded nonwoven layers, and/or the one or more hydroentangled layers that may be independently from each other comprise a synthetic polymer, such as such as a polyolefin, a polyester, a polyamide, or any combination thereof. The polyolefin, for example, may comprise a polypropylene, a polypropylene copolymer, a polyethylene, a polyethylene copolymer, or any combination thereof.

In accordance with certain embodiments of the invention, wherein the fabric has an alcohol repellency rating of at least 5 as determined according to IST 80.8, or at least about 6 as determined according to IST 80.8, or at least about 7 as determined according to IST 80.8or at least about 8 as determined according to IST 80.8.

In accordance with certain embodiments of the invention, the fabric has a static decay of from about 0.01 to about 0.5 seconds as tested according to IST 40.2 performed at 50% R.H. and using 10% remaining charge as the cut-off level, such as at least about any of the following: 0.01, 0.02, 0.05, 0.08, and 0.1 seconds and/or at most about any of the following: 5, 4, 3, 2, 1.5, 1.2, and 1 seconds.

In accordance with certain embodiments of the invention, the fabric has a static decay of from about 1 to about 3 seconds as tested according to IST 40.2 performed at 30% R.H. and using 10% remaining charge as the cut-off level, such as at least about any of the following: 1, 1.2, 1.4, 1.6, 1.8, and 2 seconds and/or at most about any of the following: 3, 2.8, 2.6, 2.4, 2.2 and 2 seconds.

In accordance with certain embodiments of the invention, the fabric has a hydrohead from about 10 mbar to about 100 mbar, such as from at least about any of the following: 10, 20, 30, 40, 50, 60, 65, 70, 75, and 80 mbar and/or at most about any of the following: 100, 95, 90, 85, and 80 mbar.

In accordance with certain embodiments of the invention, the fabric has a bonded area defined by a plurality of discrete bonding sites. For example, the bonded area comprises no more than 40%, no more than 30%, no more than 25%, no more than 20%, no more than 15%, no more than 10%, no more than 5%, no more than 3%.

In accordance with certain embodiments of the invention, the fabric may be provided in the form or at least as a component for a hygiene article, such as an absorbent underpad, a feminine hygiene article, toddler pull-ups, or diapers. As noted above, the fabric may comprise one or more discrete treated areas comprise. These one or more discrete treated areas may comprise high risk areas for exposure to bodily fluids, such as blood, urine, or stool.

In another aspect, the present invention provides an absorbent article comprising a fabric having a NFBC and/or FBC, such as those described herein, a liquid permeable topsheet, and an absorbent body located between the fabric and the liquid permeable topsheet. As noted above, the absorbent article may be an absorbent underpad, a feminine hygiene article, a toddler pull-up, or a diaper (e.g., child and adult diapers). In this regard, for example, the fabric having a NFBC and/or FBC, such as those described herein, may comprise a backsheet for at least a portion of the article. For instance, the fabric having a NFBC and/or FBC, such as those described herein, may replace the traditional use of a film-based backsheet. In accordance with certain embodiments of the invention, for example, the absorbent article may be devoid of a backsheet comprising a film.

In another aspect, the present invention provides an absorbent diaper comprising a front region, a back region, an intermediate crotch region located between the front region and the back region. The absorbent diaper may also include at least one barrier leg cuff, in which the at least one barrier leg cuff comprises a fabric having a NFBC and/or FBC, such as those described herein. For example, the at least one barrier leg cuff may include a free distal edge and a contractible cuff disposed adjacent to a longitudinal edge of the diaper. In accordance with certain embodiments of the invention, the at least one barrier leg cuff is attached at least to the crotch region of the diaper.

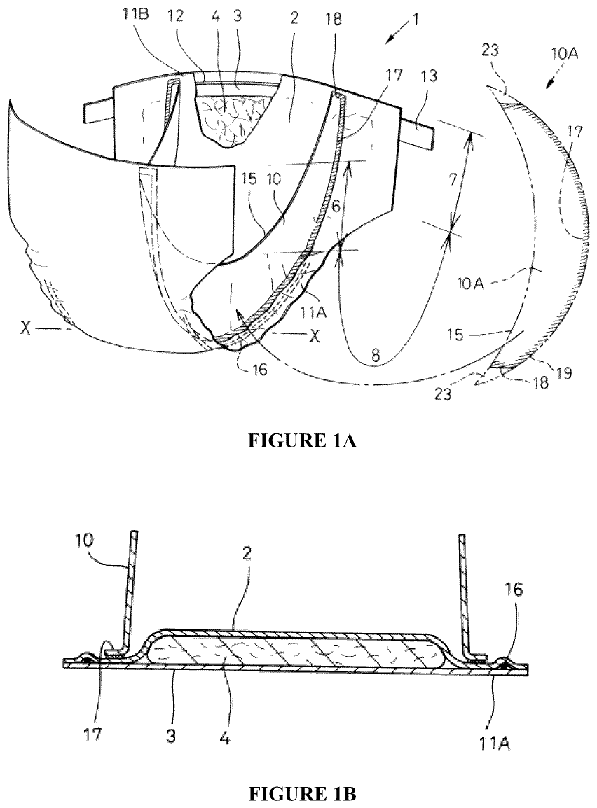

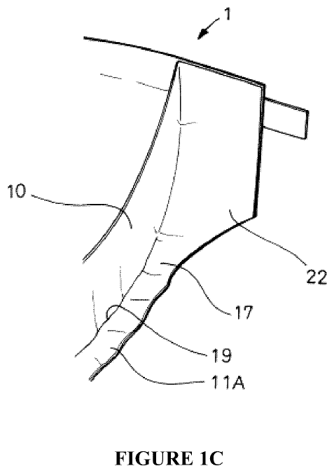

FIGS. 1A through 1C illustrate an example absorbent article (e.g., a diaper) according to certain embodiments of the invention. In this regard, the embodiments illustrated in FIGS. 1A through 1C are merely illustrative and not limiting, such as with respect to any particular structure illustrated in FIGS. 1A through 1C. FIGS. 1A and 1B illustrate an example diaper 1 according to certain embodiments of the invention, in which the diaper 1 includes a liquid-permeable topsheet 2, a liquid-impermeable backsheet 3, such as the barrier fabrics described and disclosed herein, and a liquid-absorbent core 4 sandwiched between the liquid-permeable topsheet 2 and the liquid-impermeable backsheet 3. The topsheet 2 and the backsheet 3 may outwardly extend from the periphery of the liquid-absorbent core 4 and may be bonded together in these outwardly extending regions to form leg surrounding flaps 11A and a waist surrounding flap 11B. The diaper 1 may be longitudinally composed of a front region 6, a rear region 7, and an intermediate crotch region 8 located between the front region 6 and the rear region 7. An elastic member (first elastic member) 12 may be bonded in its stretched state to, for example, the inner surface of the backsheet 3 circumferentially along the waist surrounding flap 11B of the rear region 7, and a pair of tape fasteners 13 may outwardly extend from transversely opposite side edges of the rear region 7, respectively. A pair of barrier leg cuffs 10 may longitudinally extend on the inner surface of the diaper 1 along transversely opposite (periphery) sides of the diaper 1 across the front and rear regions 6, 7 as well as the crotch region 8, in which the barrier leg cuffs 10 may bear against the wearer's crotch as the diaper 1 is worn. Each leg surrounding flap 11A may be cut away to form a circular-arc-shaped notch outside the associated barrier leg cuff 10 and an elastic member (second elastic member) 16 may be bonded in its stretched state to the inner surface of the backsheet 3 in parallel to and inside the circular arc or, parallel to the edge of the absorbent core.

Referring to FIG. 1B, the bonding margin 17 along which the barrier leg cuff 10 is bonded to the inner surface of the diaper 1 is dimensioned to be sufficiently large to cover the leg surrounding flap 11A as well as wings 22 of the front and rear regions 6, 7 and to define a substantially identical outline as that of the diaper 1.

Although FIGS. 1A through 1C illustrate a particular construction of an absorbent article according to one example embodiment of the invention, a wide range of other particular constructions incorporating a barrier nonwoven fabric as disclosed herein may be utilized. For example, certain embodiments of the invention may comprise an absorbent article as disclosed in U.S. Pat. No. 7,435,243 (disclosing an absorbent article comprising barrier leg cuffs and an elasticized outer leg cuff), in which the barrier leg cuffs may comprise a barrier nonwoven fabric as disclosed herein. The contents of U.S. Pat. No. 7,435,243 are incorporated by reference herein to the extent that such disclosure is consistent with the present disclosure. In accordance with certain embodiments of the invention, absorbent articles may comprise a wide-variety of structures including, by way of non-limiting examples, structures as described in any one of U.S. Pat. Nos. 4,738,677, 4,795,454, and 5,582,606, in which the barrier leg cuffs may comprise a barrier nonwoven fabric as disclosed herein. The contents of U.S. Pat. Nos. 4,738,677, 4,795,454, and 5,582,606 are each incorporated by reference herein to the extent that such disclosures are consistent with the present disclosure.

In yet another aspect, the present invention provides a method of forming a fabric. The method may comprise the following: (i) providing or forming a fibrous substrate including a first outermost surface and a second outermost surface; (ii) topically applying a fluorinated barrier coating (FBC) composition or a non-fluorinated barrier coating (NFBC) composition on at least a portion of at least the first outermost surface, wherein the NFBC comprises (a) a plurality of radiation curing silicones or (b) a dispersion comprising a solvent, a wax or component thereof, and a retention aid; and (iii) (a) curing the plurality of radiation curing silicones via exposure to radiation, such as ultraviolet (UV) radiation if the NFBC composition comprises the plurality of radiation curing silicones, or (b) actively or passively evaporating the solvent from the dispersion or the FBC composition to provide a fabric having a NFBC and/or FBC, such as those described herein

In accordance with certain embodiments of the invention, the plurality of radiation curing silicones comprise silicone acrylate oligomers or polymers. The NFBC composition may further comprises a photoinitiator, such as an alpha-hydroxy-ketone, and wherein curing the plurality of radiation curing silicones is conducted in an inert environment, such as nitrogen. In this regard, the silicone acrylate oligomers or polymers in the presence of the photoinitiator may begin to react and cure to form a network of the cured silicone acrylate oligomers or polymers upon exposure to radiation, such as UV radiation. Beneficially, such curing operations for silicone acrylate oligomers or polymers may be carried out at room temperature (e.g., 20-25° C.). In accordance with certain embodiments of the invention, the method may further compris a step of subjecting at least the first outermost surface to corona treatment prior to the step of topically applying the NFBC composition.

In accordance with certain embodiments of the invention, the plurality of radiation curing silicones may comprise one or more epoxy silicone oligomers or polymers. The NFBC composition may further comprises a photocatalyst that forms a strong acid that initiates curing via the epoxy groups upon exposure to radiation, such as UV radiation. When epoxy functional silicone oligomers or polymers are utilized, the curing operation may beneficially be carried out in an open environment (e.g., in the presence of oxygen).

In accordance with certain embodiments of the invention, the dispersion comprising a solvent, a wax or component thereof, and a retention aid may comprise any composition disclosed in U.S. Ser. No. 17/498,221 (i.e., U.S. Publication No. 2022/01788078), for example as a component of the NFBC, assigned to AGC Chemicals Americas, Inc. (PA, USA), the contents of which are hereby incorporated by reference to the extent its disclosure is consistent with the aims of embodiments of the present invention. In this regard, for example, the solvent may include a variety of solvating liquids or may include a single liquid. The solvent generally includes at least water. Other liquids that may optionally be included in the solvent are liquids that are miscible with water. Specific examples of the water-miscible solvent include at least one solvent selected from the group of propylene glycol, dipropylene glycol, tripropylene glycol, propylene glycol monomethyl ether, propylene glycol monoethyl ether, dipropylene glyocol monomethyl ether, dipropylene glycol monoether ether, tripropylene glycol monomethyl ether, diacetone alcohol, and combinations thereof. Most typically, the solvent includes water or a combination of water and at least one water-miscible solvent selected from the group of propylene glycol, dipropylene glycol and tripropylene glycol.

The dispersion may generally include the solvent in an amount of at least 40 parts by weight, based on 100 parts by weight of the dispersion. Alternatively, the dispersion may include the solvent in an amount of from 40 to 90, from 50 to 90, from 60 to 90, from 70 to 90, from 80 to 90, from 50 to 80, 60 to 80, or about 70, parts by weight based on 100 parts by weight of the dispersion. For example, the solvent may include water (e.g. tap water) and dipropylene glycol, with the water present in an amount of from 50 to 75 parts by weight and the dipropylene glycol present in an amount of from 15 to 40 parts by weight, each based on 100 parts by weight of the dispersion. Additionally or alternatively, the wax may be generally present within the dispersion in an amount of from 10 to 50 parts by weight, based on 100 parts by weight of the dispersion. Alternatively, the wax may be present in an amount of from 10 to 45, 10 to 40, 10 to 35, 15 to 50, 20 to 50, or 25 to 50, parts by weight based on 100 parts by weight of the dispersion. The dispersion, in accordance with certain embodiments of the invention, may include the retention aid in an amount of from 0.1 to 12 parts by weight, based on 100 parts by weight of the dispersion. Alternatively, the retention aid may be present in the dispersion in an amount of from 0.1 to 12, from 0.3 to 12, from 0.5 to 12, from 0.7 to 12, from 0.9 to 12, from 2.0 to 12, from 3.0 to 12, from 4.0 to 12, from 5.0 to 12, from 0.1 to 10, from 0.1 to 8, from 0.1 to 6, or from 0.1 to 4, parts by weight based on 100 parts by weight of the dispersion.

In accordance with certain embodiments of the invention, the step of providing the fibrous substrate may comprise providing a pre-treated fibrous substrate, wherein the pre-treated fibrous substrate optionally comprises an antistatic composition located on at least a portion of the first outermost surface, on at least a portion of the second outermost surface, or both.

In accordance with certain embodiments of the invention, the step of topically applying the NFBC composition may comprise a spraying operation, a roller coating operation, such as kiss-coating, or an offset gravure coating operation. Additionally or alternatively, the method may comprise bonding the fibrous structure, such as by a thermal bonding process or ultrasonic bonding process. For example, the bonding operation may comprise thermal calendering of the fabric or through-air-bonding.

### EXAMPLES

The present disclosure is further illustrated by the following examples, which in no way should be construed as being limiting. That is, the specific features described in the following examples are merely illustrative and not limiting.

### Example Set 1

### A. Test Methods

Basis weight of the following examples was measured according to ASTM test method D3776. The results were provided in units of mass per unit area in g/m² (gsm).

Alcohol repellency of the following examples was measured according to test method IST 80.8

Hydrohead of the following examples was measured according to standard test method IST 80.8 and ramping up the pressure at a rate of 60 mbar/min. A larger hydrohead value is more desirable for increased the barrier performance.

Air Permeability is a measure of air flow passing through a sheet under at a stated pressure differential between the surfaces of the sheet and was conducted according to ASTM D 737, Test area 38cm², Test Pressure @ 125 Pa, and is reported in ml/dm²/min. A larger air permeability value is indicative of improved comfort for surgical gown and drape applications.

Low Surface Tension Strikethrough Time (LSTST) is a test that determines the time it takes for a particular quantity of liquid discharged at a prescribed rate to fully penetrate a sample of a nonwoven fabric. The method employed herein is a modification to WSP 70.3 (05). The changes were as follows: the test liquid was a 32 mN/m surface tension liquid prepared with Triton-X-100 and distilled water.

### B. General Manufacturing Method

All base nonwoven samples were made from a polypropylene SMS nonwoven comprising at least a layer of polypropylene meltblown fibers positioned between at least two layers of polypropylene continuous spunbond fibers and point bonded using a hot calender. More specifically the inventive sample base materials were SMMMS or SMMMMS structures made on a 5 or 6 beams production line known as a Reicofil 4 at similar speed and process conditions. The process consisted of forming a plurality of continuous spunbond filaments from a first bean (e.g., beam 1) that are deposed on a foraminous moving surface, then using Beams 2, 3, 4 (e.g., for a three layer meltblown structure) or Beams 2, 3, 4, 5 (e.g., for a four layer meltblown structure) for forming polypropylene meltblown fibers that are deposited on top of the layer of continuous spunbond filaments from Beam 1. Subsequently, an additional beam (e.g., Beam 5 or Beam 6) is used to spin continuous spunbond filaments that are deposited on top of the meltblown layers to form an intermediate nonowoven composite web. The intermediate composite web is then fed to the nip point of a calender where it was point bonded under a pressure 950 N/cm and 160° C. temperature. The bonding pattern occupied about 18% of the nonwoven surface. The bond points had an elliptical shape. Inventive Examples identified as Example 3, for example, was manufactured in this method. Inventive Example 8, however, was a hydroentagled composite of wood pulp with synthetic fibers. Inventive Examples 10 and 12 were 100% PET spunlace fabrics.

Table 1 below provides a summary of the Test Result Data for Inventive Examples identified as Examples 3, 8, 10, 12, 14, which were coated with a NFBC and the Comparative Examples are identified as Examples 1, 2, 4, 5, 6, 7, 9, 11, 13, which were either untreated or C6FC treated or a breathable film as identified in Table 1.

With regard to the Inventive Examples, Example 3 was a 44 gsm SMMMS structure that was hand treated with ˜1.5% active add-on (e.g., dry basis) of the NFBC. This example achieved an average Liquid Strike Through Time (LSTST) of 529 seconds (up to 722 seconds), which compared reasonably well to the C6FC coated nonwoven of Example 1, which had the same basis weight. Example 3 utilized a 1 wt. % on a dry basis of a wetting agent and 7 wt. % on a dry basis of a cationic non-fluorochemical agent, such as those described and disclosed herein.

Example 8 was a 75 gsm EFP (e.g., hydroentangled composite of wood pulp and polypropylene continuous fibers) treated with ˜1.7% active add-on (e.g., dry basis) of the NFBC. This example achieved an average LSTST of 2372 seconds (up to 3252 second), a hydrohead of 27 mbar, an IPA 40% repellency and Spray Impact <0.1 g per AATCC 42. These performance properties are similar to C8 or C6 FC chemical treated EFP material for surgical gowns or drapes. Example 8 utilized 8 wt. % on a dry basis of a cationic non-fluorochemical agent, such as those described and disclosed herein.

Examples 10 (30 gsm) and 12 (40 gsm) were formed from 100% PET Spunlace base made in Berry Europe—Ostomy Nonwoven and treated with ˜1.7% active add-on (dry basis) of the NFBC. Each of these examples achieved average hydrohead of around 11 mbar and an LSTST 8.4 seconds. These performance properties met Berry Europe—Ostomy C6FC treated nonwoven requirement. Examples 10 and 12 utilized 8 wt. % on a dry basis of a cationic non-fluorochemical agent, such as those described and disclosed herein.

Additionally, the Inventive Examples also passed the blood drop test with synthetic blood in accordance with ASTM F1670M (e.g., 15 minutes droplets exposure without blood penetration).

In Example 14, a 13 gsm SMS nonwoven fabric was constructed that had 16% by weight of meltblown fibers (AMB 1.5), in which the SMS was treated with ˜1.5% active add-on (dry basis) of a NFBC. This example achieved an average LSTST of 35 second, which is comparable to fluorine treated diaper back sheet in the current market.

TABLE 1

| | Hydro Static | | | | | | |
|---|---|---|---|---|---|---|---|
| | Head | | Max IPA | | | | AP |
| | (60 mbar) | LSTST | Repellency | 8 W | Thickness | AP | m3/m2/ |
| Sample ID | 3rd drop | second | rating | gsm | microns | CFM | min |
| (1) 44 gsm SMMMS untreated made on S2 S1 China | 45 | 20.34 | 2 | 43.51 | 319.00 | 46.33 | 14.13 |
| (2) 44 gsm SMMMMS C6FC AR/AS treated (SZT2A215903502) | 77 | 942 | 8 | 44.73 | 356.67 | 32.63 | 9.94 |
| (3} LW722-A-1/2 (44 gsm SMMMS hand treated with | | 529 | 4 | 44 | | | |
| Non-FC Example A | | | | | | | |
| (4) 16 gsm BR172 for Cardinal breathable film | 57 | >1800 | | 16.20 | 23.33 | 0.01 | 0.00 |
| (5) 69 gsm SMMMMS [symbol] FC AR/A5 treated: SZT20216506703 | 134 | 576 | 8 | 72.21 | 470.00 | 13.50 | 4.11 |
| (for Cardinal underpad code SCD4959F) | | | | | | | |
| (6) 70 gsm SMMMMIS untreated: SZTD911612103 | 88 | 63 | 2 | 70.64 | 455.00 | 15.50 | 4.72 |
| (7) 60 gsm SMMMMS untreated: SZTD911611503 | 119 | 144 | 2 | 50.50 | 428.67 | 20.47 | 6.25 |
| (8) EFP treated with Non-FC Example A sample | 27 | 2372 | 4 | 75 | 323 | 54.9 | 16.7 |
| (9) 30 gsm 100% PET Spunlace base made in Berry Europe- | 92 | 4.6 | 1 | 30 | 444 | 562 | 202 |
| Ostomy Nonwoven | | | | | | | |
| (10) 30 gsm 100% PET Spunlace base made in Berry Europe- | 11.2 | 8.43 | 4 | 30.5 | | | |
| Ostomy Nonwoven treated with Non-FC Example A | | | | | | | |
| (11) 45 gsm 100% PET Spunlace base made in Berry Europe- | 8.3 | 4.05 | 1 | 40 | 454 | 500 | 153 |
| Ostomy Nonwoven | | | | | | | |
| (12) 40 gsm 100% PET Spunlace base made in Berry Europe- | 10.7 | 8.54 | 4 | 40.5 | | | |
| Ostomy Nonwoven treated with Non-FC Example A | | | | | | | |
| (13) 13 gsm Hygiene with 16% MB [AMB 1.5] | 18.79 | 11.27 | | 13 | | | 42.81 |
| (14) 13 gsm Hygiene with 16% MB [AMB 1.5] treated with | | 535 | 3 | 13 | | | |
| Non-FC Example A | | | | | | | |

[symbol]

indicates data missing or illegible when filed

### Example Set 2

An additional set of examples were conducted and tested for various physical properties. For example, a 45 gsm PP SMMMS KamiSoft material and 44 gsm, 69 gsm PP SMMMMS nonwovens were produced and coated with Non-FC Example A plus a higher percentage of binder compared to that of Examples 8, 11 and 13 from Example Set 1. Test results are listed in Table 2 below. These coated samples with higher % binder (e.g., on a dry basis) have 1.76 wt % solid Non-FC Example A chemical with 1.6 wt % binder (ration on a dry basis of Non-FC Example A (excluding binder) to binder of 1.1:1). The results show that the Ethanol Alcohol repellency achieved up to 70% repellency with the test method IST 80.8. Also the treated nonwovens maintained MD tensile strength after finishing process (tested by Berry MONC lab Test Code 56: pull speed 30 cm/min, jaw separation of 10 cm, sample size of 50 mm×175 mm, grip size of 50 mm, and units in N/5 cm). The static decay was tested using the standard test method IST 40.2 performed at 50% RH using 50% remaining charge as cut-off level. The properties of the resulting fabrics (Examples 8, 11 and 13) are summarized in Table 2 below. A static decay at 50% RH with 50% cut-off need to be less than 1 second with Target 0.5 second.

The binder used was a self-crosslinking non-ionic acrylic binder emulsion, and the observed IPA (Isopropanol repellent) rating improved from 4 to 5 (40% to 50% IPA repellent) and Ethanol repellency rating improved from 5 to 7 (50% to 70% IPA repellent) by add higher amount of binder. Additionally, 0.8%˜1.6% by weight on a dry basis of non-FC formula was added for 30 gsm, 40 gsm 100% PET Spunlace nonwoven and 40 gsm PE spunbond, PP SMMMMS nonwoven.

The Sample (2) 40 gsm PE spunbond (medical Ostomy nonwoven) treated with ˜1.32% dry weight add-on (=1.32 wt %) Non-FC Example A plus 1.2% Binder achieved IPA 50% repellency and 70% Ethanol Alcohol repellency. Example 2 utilized a 1.2 wt. % on a dry basis of a wetting agent, 6 wt. % on a dry basis of a cationic non-fluorochemical agent, such as those described and disclosed herein, and 3 wt. % on a dry basis of binder, as noted below.

The Sample (4), (6) 30 gsm & 40 gsm 100% PET Spunlace (medical Ostomy nonwoven) treated with ˜1.32% dry weight add-on (=1.32 wt %) Non-FC Example A plus 0.8% Binder achieved IPA 50% repellency and 70% , 80% Ethanol Alcohol repellency. These examples utilized a 1.2 wt. % orn a dry basis of a wetting agent, 6 wt. % on a dry basis of a cationic non-fluorochemical agent, such as those described and disclosed herein, and 2 wt. % on a dry basis of binder, as noted below.

The Samples (8), (11), and (23) were 45 gsm, 44 gsm, and 69 gsm polypropylene SMMMS nonwovens, respectively. Each Sample utilized a 1.6 wt. % on a dry basis of a wetting agent, 8 wt. % on a dry basis of a cationic non-fluorochemical agent, such as those described and disclosed herein, and 4 wt. % on a dry basis of binder, as noted below.

In the foregoing inventive examples, the Non-FC Example A was a cationic acrylic based polymer, containing an alkyl silane methacrylate group. The wetting agent mentioned above was Alkanol™ 6112 (Decan-1-ol; CAS No. 112-30-1).

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A fabric, comprising:
(i) a fibrous substrate including a first outermost surface and a second outermost surface; and
(ii) a non-fluorinated barrier coating (NFBC) located on at least a portion of a first outermost surface of the fabric, at least a portion of a second outermost surface of the fabric, or both and/or a fluorinated barrier coating (FBC) located on at least a portion of a first outermost surface of the fabric, at least a portion of a second outermost surface of the fabric, or both.

2. The fabric of claim 1, wherein the fabric includes the FBC, and wherein the FBC comprises at least one fluorochemical comprising at least one C4 fluorochemical, a C6 fluorochemical, a C8 fluorochemical, a C10 fluorochemical, or any combination thereof.

3. The fabric of claim 2, wherein the FBC comprises a digitally printed or sprayed composition comprising a plasma cured composition.

4. The fabric of claim 1, wherein the fabric includes the NFBC, and wherein the NFBC comprises a first NFBC comprising a first NFBC composition located on the first outermost surface of the fabric and a second NFBC comprising a second NFBC composition located on the second outermost surface, and wherein the first NFBC composition and the second NFBC composition are identical to each other or different from each other.

5. The fabric of claim 1, wherein the NFBC comprises a cured siliconized coating comprising a network of radiation cured silicones.

6. The fabric of claim 5, wherein the network of radiation cured silicones comprises the reaction product of one or more silicone acrylate oligomers or polymers or one or more epoxy silicone oligomers or polymers.

7. The fabric of claim 6, wherein the one or more silicone acrylate oligomers or polymers are selected from Formula (I): 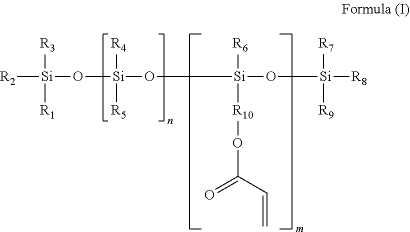
wherein,
R1-R9 are independently selected from —H, a C1-C10 radical, —OH, alkoxy radical, and an acrylate functional group,
R10 is selected from a C1-C10 hydrocarbon;
n is selected from 1 to about 100, and
m is selected from 1 to about 100.

8. The fabric of claim 6, wherein the one or more epoxy silicone oligomers or polymers are selected from Formula (II): 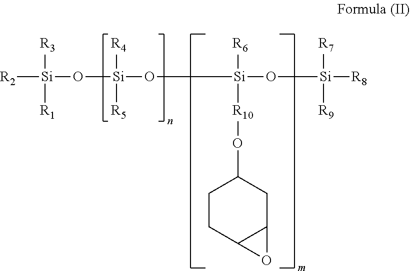
wherein
R1-R9 are independently selected from —H, a C1-C10 radical, —OH, alkoxy radical, and an acrylate functional group,
R10 is selected from a C1-C10 hydrocarbon;
n is selected from 1 to about 100, and
m is selected from 1 to about 100.

9. The fabric of claim 1, wherein the NFBC comprises (a) a wax or a component thereof having an acid value of from 10 mg to 220 mg, KOH/g as measured in accordance with USP 401, such as at least about any of the following: 10, 15, 20, 30, 40, 50, 60, 80, and 100 mg, KOH/g as measured in accordance with USP 401 and/or at most about any of the following: 220, 200, 180, 160, 150, 140, 120, and 100 mg, KOH/g as measured in accordance with USP 401 and (b) a retention aid comprising a nitrogen-containing polymer independently selected from the group consisting of:
(i) a nitrogen-containing polymer of Formula (III), 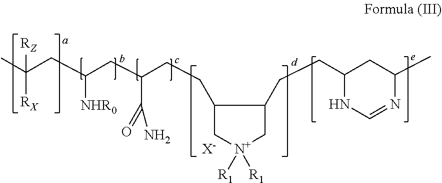
wherein ‘a’, ‘b, ‘c’, ‘d’, and ‘e’ individually represent the molar percent of each repeating unit included in the nitrogen-containing polymer of Formula (III),
wherein R₀ is independently selected from the group consisting of: of H, 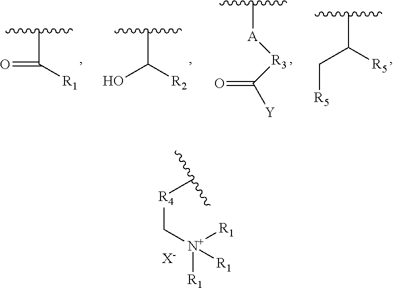
and combinations thereof, and
wherein:
R_{z} is independently selected from H, —CH₃, and combinations thereof,
Rₓ is independently selected from H, —OH, —COOH, —COOR₁, —OCOR₁, —R₁, —R₃OH, —OR₁, —NR₁R₁, —R₃NH₂, —NH₂, —COO(CH₂)₂N(R₁)₂, —COO(CH₂)₂N+(R₁)₃X⁻, —COO(CH₂)₃N+(R₁)₃X⁻, and combinations thereof, with the proviso that when Rₓ is —NH₂, R_{z} is —CH₃,
Y is independently selected from H, —OH, —R₁, —OR₁, —NR₁R₁, —NH₂, and combinations thereof,
Z is independently selected from H, —OH, —C═O, —R₁, —OR₁, —NR₁R₁, —NH2 and combinations thereof,
R₁ is independently selected from H, a straight chain or branched alkyl or alkenyl containing up to 22 carbons, and combinations thereof,
R₂ is independently selected from H, a monosaccharide, an oligosaccharide, polysaccharide moiety, a straight or branched alkyl or alkenyl group up to 22 carbons optionally containing a hydroxyl or aldehyde group, and combinations thereof,
R₃ is independently selected from a straight chain or branched alkyl or alkenyl containing up to 22 carbons or combinations thereof,
R₄ is independently selected from a straight chain or branched alkyl group containing up to 18 carbons, optionally substituted with a hydroxyl group, and combinations thereof,
R₅ is independently selected from H, —OH, —COOH, —COOR₁, —OCOR₁, —R₁, —R₁OH, —OR₁, —CONH₂, —CONHCHOHCHO, —NR₁, —NR₁R₁, —R₁NH₂, —NH₂, and combinations thereof,
A is independently selected from C═O, —CH₂, and combinations thereof, and
X⁻ is independently an anion;
ii. a polyethyleneimine;
iii. a polyaminoamide;
iv. a copolymer formed from the reaction product of epichlorohydrin and dimethylamine;
and
v. combinations thereof.

10. The fabric of claim 9, wherein ‘a’, ‘b, ‘c’, ‘d’, and ‘e’ each independently from each other have a value from 0 to 100 mol. %.

11. The fabric of claim 1, wherein the NFBC comprises from about 0.05 to about 10% by weight on a dry basis of the fabric.

12. The fabric of claim 1, wherein the fibrous substrate comprises one or more spunbond layers, one or more meltblown layers, one or more melt-fibrillated layers, one or more electrospun layers, one or more carded nonwoven layers, one or more hydroentangled layers, or any combinations thereof.

13. The fabric of claim 1, wherein the fabric further comprises at least one binder.

14. An absorbent article, comprising:
(i) a fabric according to claim 1;
(ii) a liquid permeable topsheet; and
(ii) an absorbent body located between the fabric and the liquid permeable topsheet.

15. The absorbent article of claim 14, wherein the absorbent article is a diaper or a feminine-hygiene article.

16. An absorbent diaper, comprising:
a front region, a back region, an intermediate crotch region located between the front region and the back region, and at least one barrier leg cuff, wherein the at least one barrier leg cuff comprises a fabric according to claim 1.

17. The absorbent diaper of claim 16, wherein the at least one barrier leg cuff includes a free distal edge and a contractible cuff disposed adjacent to a longitudinal edge of the diaper.

18. The absorbent diaper of claim 16, wherein the at least one barrier leg cuff is attached at least to the crotch region of the diaper.

19. A method of forming a fabric, comprising:
(i) providing or forming a fibrous substrate including a first outermost surface and a second outermost surface;
(ii) topically applying a fluorinated barrier coating (FBC) composition or a non-fluorinated barrier coating (NFBC) composition on at least a portion of at least the first outermost surface, wherein the NFBC comprises (a) a plurality of radiation curing silicones or (b) a dispersion comprising a solvent, a wax or component thereof, and a retention aid; and
(iii) (a) curing the plurality of radiation curing silicones via exposure to radiation, such as ultraviolet (UV) radiation if the NFBC composition comprises the plurality of radiation curing silicones, or (b) actively or passively evaporating the solvent from the dispersion or the FBC composition.

20. The method of claim 19, further comprising a step of subjecting at least the first outermost surface to corona treatment prior to the step of topically applying the NFBC composition.
